SEQUENCE LISTING (158 sequences)

SEQ ID NO: 1 - DNA, 15 bp, Homo sapiens
cccaggaagttttct

SEQ ID NO: 2 - DNA, 15 bp, Homo sapiens
gccaccagacggcct

SEQ ID NO: 3 - DNA, 15 bp, Homo sapiens
gcccaagagttccaa

SEQ ID NO: 4 - DNA, 15 bp, Homo sapiens
tgtgtcctgccttgt

SEQ ID NO: 5 - DNA, 15 bp, Homo sapiens
acagaaaggctccgt

SEQ ID NO: 6 - DNA, 15 bp, Homo sapiens
ctccgtgagaatcac

SEQ ID NO: 7 - DNA, 15 bp, Homo sapiens
tgatttgtgtggtct

SEQ ID NO: 8 - DNA, 15 bp, Homo sapiens
ttatctgtgaatctg

SEQ ID NO: 9 - DNA, 15 bp, Homo sapiens
aaaagattagtggct

SEQ ID NO: 10 - DNA, 15 bp, Homo sapiens
ttggtgtctgttcag

SEQ ID NO: 11 - DNA, 15 bp, Homo sapiens
ttttttattacacta

SEQ ID NO: 12 - DNA, 15 bp, Homo sapiens
agaaaaatggcatgc

SEQ ID NO: 13 - DNA, 15 bp, Homo sapiens
gcagattactttaaa

SEQ ID NO: 14 - DNA, 15 bp, Homo sapiens
gcagagatgggaagt

SEQ ID NO: 15 - DNA, 15 bp, Homo sapiens
cttcactgattcctg

SEQ ID NO: 16 - DNA, 15 bp, Homo sapiens
cattaacctaattgc

SEQ ID NO: 17 - DNA, 15 bp, Homo sapiens
tggtgagaaaaagat

SEQ ID NO: 18 - DNA, 15 bp, Homo sapiens
gagtctttgttccag

SEQ ID NO: 19 - DNA, 15 bp, Homo sapiens
gtacaccattggaga

SEQ ID NO: 20 - DNA, 15 bp, Homo sapiens
gaagcagtggatcag

SEQ ID NO: 21 - DNA, 15 bp, Homo sapiens
cccctgtttgcagtg

SEQ ID NO: 22 - DNA, 15 bp, Homo sapiens
gtgacattggtagca

SEQ ID NO: 23 - DNA, 15 bp, Homo sapiens
atcccccgggacccc

SEQ ID NO: 24 - DNA, 15 bp, Homo sapiens
gggcaggactctgct

SEQ ID NO: 25 - DNA, 15 bp, Homo sapiens
tccaggaaggtcggc

SEQ ID NO: 26 - DNA, 15 bp, Homo sapiens
ggcaaggcgagaatc

SEQ ID NO: 27 - DNA, 15 bp, Homo sapiens
ggtggtgggaaaaga

SEQ ID NO: 28 - DNA, 15 bp, Homo sapiens
gttgaaagtatgaga

SEQ ID NO: 29 - DNA, 15 bp, Homo sapiens
agctggagaaagtac

SEQ ID NO: 30 - DNA, 15 bp, Homo sapiens
accgtgggtggccat

SEQ ID NO: 31 - DNA, 15 bp, Homo sapiens
cctcatctgaaatcc

SEQ ID NO: 32 - DNA, 15 bp, Homo sapiens
tatttacagattctg

SEQ ID NO: 33 - DNA, 15 bp, Homo sapiens
gctggactgacaacc

SEQ ID NO: 34 - DNA, 15 bp, Homo sapiens
gttcctctgctcaaa

SEQ ID NO: 35 - DNA, 15 bp, Homo sapiens
aaaagaccggtgcag

SEQ ID NO: 36 - DNA, 15 bp, Homo sapiens
gagggtcagacctgc

SEQ ID NO: 37 - DNA, 15 bp, Homo sapiens
aaacagcgtggtggt

SEQ ID NO: 38 - DNA, 15 bp, Homo sapiens
ccatttggatagctt

SEQ ID NO: 39 - DNA, 15 bp, Homo sapiens
aatgagcccgctttc

SEQ ID NO: 40 - DNA, 15 bp, Homo sapiens
aatgagccttagcta

SEQ ID NO: 41 - DNA, 15 bp, Homo sapiens
tgaggactgtgttca

SEQ ID NO: 42 - DNA, 15 bp, Homo sapiens
gagataccttgacct

SEQ ID NO: 43 - DNA, 15 bp, Homo sapiens
ctggccctactcctc

SEQ ID NO: 44 - DNA, 15 bp, Homo sapiens
tttccccaggaaccc

SEQ ID NO: 45 - DNA, 15 bp, Homo sapiens
cgtgtcgttggcaga

SEQ ID NO: 46 - DNA, 15 bp, Homo sapiens
gaacgtgttattggc

SEQ ID NO: 47 - DNA, 15 bp, Homo sapiens
ccccctgaatccaga

SEQ ID NO: 48 - DNA, 15 bp, Homo sapiens
cgtgtcgttggcaga

SEQ ID NO: 49 - DNA, 15 bp, Homo sapiens
aaggtggcaatttta

SEQ ID NO: 50 - DNA, 15 bp, Homo sapiens
aacttcagtggatcc

SEQ ID NO: 51 - DNA, 15 bp, Homo sapiens
ctggccctactcctc

SEQ ID NO: 52 - DNA, 15 bp, Homo sapiens
cgtgtcgttggcaga

SEQ ID NO: 53 - DNA, 15 bp, Homo sapiens
aggaaaatggatttg

SEQ ID NO: 54 - DNA, 15 bp, Homo sapiens
aagaaccgggtctct

SEQ ID NO: 55 - DNA, 15 bp, Homo sapiens
aaaaaggattaggct

SEQ ID NO: 56 - DNA, 15 bp, Homo sapiens
tgtgtgctctaagtg

SEQ ID NO: 57 - DNA, 15 bp, Homo sapiens
ttctgcatgtgtaat

SEQ ID NO: 58 - DNA, 15 bp, Homo sapiens
tctccgtctccacct

SEQ ID NO: 59 - DNA, 15 bp, Homo sapiens
gaacctgtgcatagt

SEQ ID NO: 60 - DNA, 15 bp, Homo sapiens
ctggtgaccccatcc

SEQ ID NO: 61 - DNA, 15 bp, Homo sapiens
tgagcacggatgacc

SEQ ID NO: 62 - DNA, 15 bp, Homo sapiens
ctggtgaccccatcc

SEQ ID NO: 63 - DNA, 15 bp, Homo sapiens
acccccaagacgccc

SEQ ID NO: 64 - DNA, 15 bp, Homo sapiens
tctccgtctccacct

SEQ ID NO: 65 - DNA, 15 bp, Homo sapiens
gcgaggcgatggtga

SEQ ID NO: 66 - DNA, 15 bp, Homo sapiens
ggacacggccgaccg

SEQ ID NO: 67 - DNA, 15 bp, Homo sapiens
gtgacccgcggcgag

SEQ ID NO: 68 - DNA, 15 bp, Homo sapiens
acgctactcaccagg

SEQ ID NO: 69 - DNA, 15 bp, Homo sapiens
tggagcaggggtgac

SEQ ID NO: 70 - DNA, 15 bp, Homo sapiens
gatcctacctccgga

SEQ ID NO: 71 - DNA, 15 bp, Homo sapiens
tctccgtctccacct

SEQ ID NO: 72 - DNA, 15 bp, Homo sapiens
ccactcctgtgggtg

SEQ ID NO: 73 - DNA, 15 bp, Homo sapiens
gaacctgtgcatagt

SEQ ID NO: 74 - DNA, 15 bp, Homo sapiens
ctggtgaccccatcc

SEQ ID NO: 75 - DNA, 16 bp, Homo sapiens
agagatggaggtgaga

SEQ ID NO: 76 - DNA, 15 bp, Homo sapiens
acgctactcaccagg

SEQ ID NO: 77 - DNA, 15 bp, Homo sapiens
tctccgtctccacct

SEQ ID NO: 78 - DNA, 15 bp, Homo sapiens
ctggtgaccccatcc

SEQ ID NO: 79 - DNA, 15 bp, Homo sapiens
ctctgcacttgcggc

SEQ ID NO: 80 - DNA, 15 bp, Homo sapiens
tctccgtctccacct

SEQ ID NO: 81 - DNA, 15 bp, Homo sapiens
gaacctgtgcatagt

SEQ ID NO: 82 - DNA, 15 bp, Homo sapiens
ctggtgaccccatcc

SEQ ID NO: 83 - DNA, 15 bp, Homo sapiens
actgcccaggctggg

SEQ ID NO: 84 - DNA, 15 bp, Homo sapiens
tctccgtctccacct

SEQ ID NO: 85 - DNA, 15 bp, Homo sapiens
gaacctgtgcatagt

SEQ ID NO: 86 - DNA, 15 bp, Homo sapiens
ctggtgaccccatcc

SEQ ID NO: 87 - DNA, 15 bp, Homo sapiens
acgctactcaccagg

SEQ ID NO: 88 - DNA, 15 bp, Homo sapiens
ccactcctgtgggtg

SEQ ID NO: 89 - DNA, 15 bp, Homo sapiens
gaacctgtgcatagt

SEQ ID NO: 90 - DNA, 15 bp, Homo sapiens
ctggtgaccccatcc

SEQ ID NO: 91 - DNA, 15 bp, Homo sapiens
caacctgtctgcatg

SEQ ID NO: 92 - DNA, 15 bp, Homo sapiens
cccatcatccagatc

SEQ ID NO: 93 - DNA, 15 bp, Homo sapiens
cgcgtggcgcgagca

SEQ ID NO: 94 - DNA, 15 bp, Homo sapiens
gaacctgtgcatagt

SEQ ID NO: 95 - DNA, 15 bp, Homo sapiens
ctggtgaccccatcc

SEQ ID NO: 96 - DNA, 15 bp, Homo sapiens
tctccgtctccacct

SEQ ID NO: 97 - DNA, 14 bp, Homo sapiens
agctgctgagcaca

SEQ ID NO: 98 - DNA, 15 bp, Homo sapiens
gaacctgtgcatagt

SEQ ID NO: 99 - DNA, 15 bp, Homo sapiens
ctggtgaccccatcc

SEQ ID NO: 100 - DNA, 15 bp, Homo sapiens
ctcaggaggggactg

SEQ ID NO: 101 - DNA, 15 bp, Homo sapiens
ctcaggaggggactg

SEQ ID NO: 102 - DNA, 15 bp, Homo sapiens
aggagggatgaagga

SEQ ID NO: 103 - DNA, 15 bp, Homo sapiens
agggaccgaaggagg

SEQ ID NO: 104 - DNA, 15 bp, Homo sapiens
gaacctgtgcatagt

SEQ ID NO: 105 - DNA, 15 bp, Homo sapiens
ctggtgaccccatcc

SEQ ID NO: 106 - DNA, 14 bp, Homo sapiens
gagacctgaggcct

SEQ ID NO: 107 - DNA, 15 bp, Homo sapiens
aagagattgtgaggg

SEQ ID NO: 108 - DNA, 15 bp, Homo sapiens
gtgaccactgacggc

SEQ ID NO: 109 - DNA, 15 bp, Homo sapiens
ctggtacttggacca

SEQ ID NO: 110 - DNA, 15 bp, Homo sapiens
atttttatatccctc

SEQ ID NO: 111 - DNA, 15 bp, Homo sapiens
gaacctcaaacaatt

SEQ ID NO: 112 - DNA, 15 bp, Homo sapiens
ggtgatgaatccctt

SEQ ID NO: 113 - DNA, 15 bp, Homo sapiens
gtgctgagaaatata

SEQ ID NO: 114 - DNA, 15 bp, Homo sapiens
atttttatatccctc

SEQ ID NO: 115 - DNA, 15 bp, Homo sapiens
agaaaccagggtggg

SEQ ID NO: 116 - DNA, 15 bp, Homo sapiens
cagcctccggtgcct

SEQ ID NO: 117 - DNA, 15 bp, Homo sapiens
gtgcccacacctggt

SEQ ID NO: 118 - DNA, 15 bp, Homo sapiens
cttagaatctcaact

SEQ ID NO: 119 - DNA, 15 bp, Homo sapiens
tcaagtaaccgctgc

SEQ ID NO: 120 - DNA, 15 bp, Homo sapiens
atcgaccaggagatc

SEQ ID NO: 121 - DNA, 15 bp, Homo sapiens
gacacccagcgcttc

SEQ ID NO: 122 - DNA, 15 bp, Homo sapiens
ctgggcccccctgtc

SEQ ID NO: 123 - DNA, 15 bp, Homo sapiens
gccctgctgtcaccc

SEQ ID NO: 124 - DNA, 15 bp, Homo sapiens
tgtcggctagcacat

SEQ ID NO: 125 - DNA, 15 bp, Homo sapiens
ctcggcttgaattga

SEQ ID NO: 126 - DNA, 15 bp, Homo sapiens
tatacacacatctcc

SEQ ID NO: 127 - DNA, 15 bp, Homo sapiens
gggcttcatggacct

SEQ ID NO: 128 - DNA, 15 bp, Homo sapiens
agggaaggtgtctca

SEQ ID NO: 129 - DNA, 15 bp, Homo sapiens
gaagatgcctcagta

SEQ ID NO: 130 - DNA, 15 bp, Homo sapiens
cggggcctcagtggg

SEQ ID NO: 131 - DNA, 15 bp, Homo sapiens
gggctgccggtctac

SEQ ID NO: 132 - DNA, 15 bp, Homo sapiens
cgccatctgacctga

SEQ ID NO: 133 - DNA, 15 bp, Homo sapiens
gcatgatcacgggag

SEQ ID NO: 134 - DNA, 15 bp, Homo sapiens
tgatcgtgggaggga

SEQ ID NO: 135 - DNA, 15 bp, Homo sapiens
acactgtgcgccacc

SEQ ID NO: 136 - DNA, 15 bp, Homo sapiens
ctgtgtaccacctgc

SEQ ID NO: 137 - DNA, 15 bp, Homo sapiens
ccagaccaagaagga

SEQ ID NO: 138 - DNA, 15 bp, Homo sapiens
cctgctgtgacctga

SEQ ID NO: 139 - DNA, 15 bp, Homo sapiens
tacacgcgggacaga

SEQ ID NO: 140 - DNA, 15 bp, Homo sapiens
gggcttcatgaatga

SEQ ID NO: 141 - DNA, 15 bp, Homo sapiens
gctggtgctgtgtgg

SEQ ID NO: 142 - DNA, 15 bp, Homo sapiens
agcgctgcgtcttct

SEQ ID NO: 143 - DNA, 15 bp, Homo sapiens
gtggggcactccctg

SEQ ID NO: 144 - DNA, 15 bp, Homo sapiens
ggacttcgtggttca

SEQ ID NO: 145 - DNA, 15 bp, Homo sapiens
caggcccagcctgat

SEQ ID NO: 146 - DNA, 15 bp, Homo sapiens
ccacatgaccaaggt

SEQ ID NO: 147 - DNA, 15 bp, Homo sapiens
tggtgggggctgagc

SEQ ID NO: 148 - DNA, 15 bp, Homo sapiens
gtggggcactccctg

SEQ ID NO: 149 - DNA, 15 bp, Homo sapiens
tagaggaacattagt

SEQ ID NO: 150 - DNA, 15 bp, Homo sapiens
agttatgtctactta

SEQ ID NO: 151 - DNA, 15 bp, Homo sapiens
tagaggaacattagt

SEQ ID NO: 152 - DNA, 15 bp, Homo sapiens
agttatgtctactta

SEQ ID NO: 153 - DNA, 15 bp, Homo sapiens
gatacaataattttt

SEQ ID NO: 154 - DNA, 15 bp, Homo sapiens
agttatgtctactta

SEQ ID NO: 155 - DNA, 15 bp, Homo sapiens
tagaggaacattagt

SEQ ID NO: 156 - DNA, 15 bp, Homo sapiens
tcactcacgttcatg

SEQ ID NO: 157 - DNA, 15 bp, Homo sapiens
tgcgtgcagaagggc

SEQ ID NO: 158 - DNA, 15 bp, Homo sapiens
cagcatctgcccttt

(19) United States
(12) Patent Application Publication
(10) Pub. No.: US 2005/0037366 A1
(43) Pub. Date: Feb. 17, 2005

(54) Individual drug safety

(76) Inventors: Joseph Gut, Grellingen (CH); Hubert Hug, Staufen (DE); Richard Schindler, Schliengen (DE); Robert Dannecker, Luzern (CH); Dario Bagatto, Allschwil (CH)

Correspondence Address: DAVID TOREN, ESQ.;SIDLEY, AUSTIN, BROWN & WOOD, LLP, 787 SEVENTH AVENUE, NEW YORK, NY, 10019-6018, US

(21) Appl. No.: 10/641,960

(22) Filed: Aug. 14, 2003

(51) Int. Cl. C12Q 1/68; G06F 19/00; G01N 33/48; G01N 33/50

(52) U.S. Cl. 435/6; 702/20

(57) ABSTRACT

The invention provides means to determine the predisposition of individuals to adverse drug reactions (ADRs). The methods are based on genotyping or parallelized enzyme and protein profiling or both. Parallelized enzyme activity profiling can be used for drug screening and development. As examples of the invention we show the prediction of adverse drug reactions of pulmonary hypertension patients by identifying genes and alleles linked to known ADRs and liver enzyme reaction profiling with ADR correlation.

### BACKGROUND OF THE INVENTION

1. Field of the Invention

Adverse Drug Reaction(ADR)-profiles are predicted by knowledge management. Genotyping and protein analysis are 10 correlated with clinical parameters.

1. Description of the Prior Art

Current technology is disclosed in U.S. Patent 15 Application 20030004202 (Elliott, J. D., Weinstock, J., Xiang, J.-N., concerning ET receptors), U.S. Patent Application 20030004199 (Ounis, I., concerning Method for preventing or treating pulmonary inflammation by administering an endothelin antagonist) and in U.S. Patent Application 20020193307 (Banting, J. D., Heaton, J. P. W., Adams, M. A. concerning Antagonism of endothelin actions).W0200292813-A1 (Matsushta Electric Ind Co Ltd, Biomolecular chip having immobilized polynucleotides or proteins for examination of biological samples and disease diagnosis), W0200290573-A2 (Infineon Technologies, biochip and other fundamental biomolecular investigations, comprises a substrate, sensor and a spaced, conductive permeation layer held at electrical potential use).

Data arising from genotyping, mRNA expression analysis and protein profiling are currently not directly linked with each other and to databases containing information on ADRs, drugs and compounds, pharmacokinetics and pharmacodynamics. Efficient algorithms for sequence multi-alignment and protein structure determination are still under development. No common system is available for the analysis of diverse data such as sequences, compounds (including similarity searches and visualization), ADRs, clinical endpoints, pharmacokinetics, pharmacodynamics etc. SafeBase™ will be developed for such purposes.

The person-to-person variability of a drug response is a major problem in clinical practice and in drug development. It can lead to both adverse effects of drugs or to therapeutic failure in individual patients or in sub-populations of patients (Meyer, U. A. & Gut, J. Genomics and the prediction of xenobiotic toxicity. Toxicology 181-182, 15 463-466 (2002).

### SUMMARY OF THE INVENTION

A major prerequisite of the invention is the availability of large parts of sequences of the human genome. Especially important are allelic variants or polymorphisms, which can be correlated to occurrences of diseases or to the susceptibility of diseases and ADRs. The main technology, developed for the study of genomics, are DNA microarrays or DNA chips. This technology is disclosed in Pennie, W. D. Custom cDNA microarrays; technologies and applications. Toxicology 181-182, 551-554 (2002); Salter, A. H. & Nilsson, K. C. Informatics and multivariate analysis of toxicogenomics data. Curr Opin Drug Discov Devel 6, 117-122 (2003); Bunney, W. E. et al. Microarray technology: a review of new strategies to discover candidate vulnerability genes in psychiatric disorders. Am J Psychiatry 160, 657-666 (2003); Simon, R., Radmacher, M. D., Dobbin, K. & McShane, L. M. Pitfalls in the use of DNA microarray data for diagnostic and prognostic classification. J Natl Cancer Inst 95, 14-18 (2003); Cheek, D. J. & Cesan, A. Genetic predictors of cardiovascular disease: the use of chip technology. J Cardiovasc Nurs 18, 50-56 (2003); Yeatman, T. J. The future of clinical cancer management: one tumor, one chip. Am Surg 69, 41-44 (2003). DNA chips are either used to study Mrna expression patterns or the detection of single nucleotide polymorphisms (SNPs). Approximately 200000 SNPs, which may directly contribute to disease, are mainly located in protein-coding regions of a gene. Currently an effort to genotype 10 million human SNPs is undertaken. Protein microarrays or protein chips have been developed to study proteomics, the analysis of large scale protein expression and function (Templin, M. F. et al. Protein microarray technology. Drug Discov Today 7, 815-822, 2002; Kusnezow, W. & Hoheisel, J. D. Antibody microarrays: promises and problems. Biotechniques Suppl, 14-23, 2002; Kusnezow, W., Jacob, A., Walijew, A., Diehl, F. & Hoheisel, J. D. Antibody microarrays: An evaluation of production parameters. Proteomics 3, 254-264, 2003). The probes are either antibodies or peptide antigens. The technique can be improved to detect protein interactions and enzyme activity. Alternative techniques to microarrays are microbeads, where the oligonucleotide or peptide is attached to a bead surface, or mass spectrometry.

The biggest current challenge is the data mining of all available sequence, mRNA and protein expression, enzyme activity, and protein structure information and integration into knowledge-management systems (e.g. SafeBase™ of TheraSTrat AG, CH-Allschwil). SafeBase™ consists of interconnected databases (e.g. compounds, clinical end points, ADRs, enzymatic pathways), which has been developed to include the future genomics and proteomics databases.

All available information on chemical and biological parameters are analyzed and clustered. This creates the possibility for chemical and biological similarity searches with multiple parameters to predict ADRs.

According to the invention Genotypes and/or enzyme and protein profiles are correlated with information on ADRs. This leads to an individual profile for the patient's susceptibility against drugs and medical treatments.

### BRIEF DESCRIPTION OF THE DRAWINGS

The invention is illustrated in more detail below with references to the drawing, wherein the drawings show:

FIG. 1: A strategy of individual drug safety;

FIG. 2: A representation of the relations of allelic and protein variants of ETB (EDNRB) with the SafeBase™ Intelligent Knowledge Browser;

FIG. 3: Potential cytotoxic mechanisms for quinones and radicals.

### DETAILED DESCRIPTION OF THE PREFERRED EMBODIMENTS

The present invention will be described with reference to the drawings, tables, and examples.

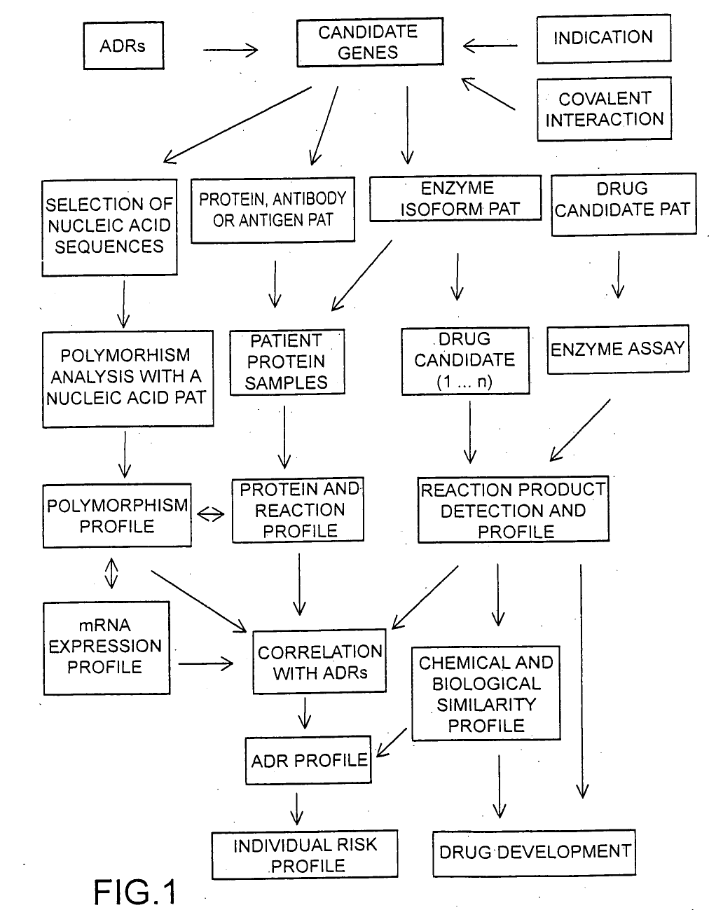

FIG. 1 shows a strategy of individual drug safety wherein data obtained by novel genomics and proteomics methods are used for the generation of ADR profiles. A surface based enzyme assay is introduced for reaction product profiling. Alternatively the drug candidates are attached to a chip surface or used in soluble form in reaction chambers on a chip. These in vitro data are correlated with ADRs, which help to predict the composition and structure of compounds with potentially fewer or no ADRs. With this sysem drug candidates can be scanned either in combination or sequentially. A pattern analysis tool (pat) is a structure for the attachment, holding and/or other format of analysis of biomolecules, e.g. microarrays, microfluidics, beads, mass spectrometry, chromatography etc.

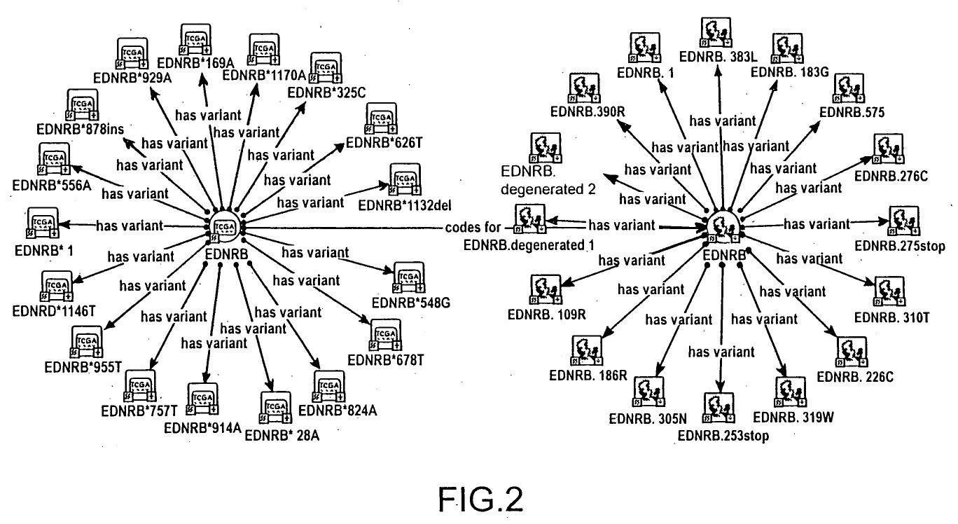

FIG. 2 shows a representation of the relations of allelic and protein variants of ET_{B} (EDNRB) with the SafeBase™ Intelligent Knowledge Browser with allelic and protein variants of ET_{B} (EDNRB) being visualized as rectangular nodes, which are connected to circular generic nodes. For clarity, the connection between the allelic and protein variant is only shown for the generic nodes.

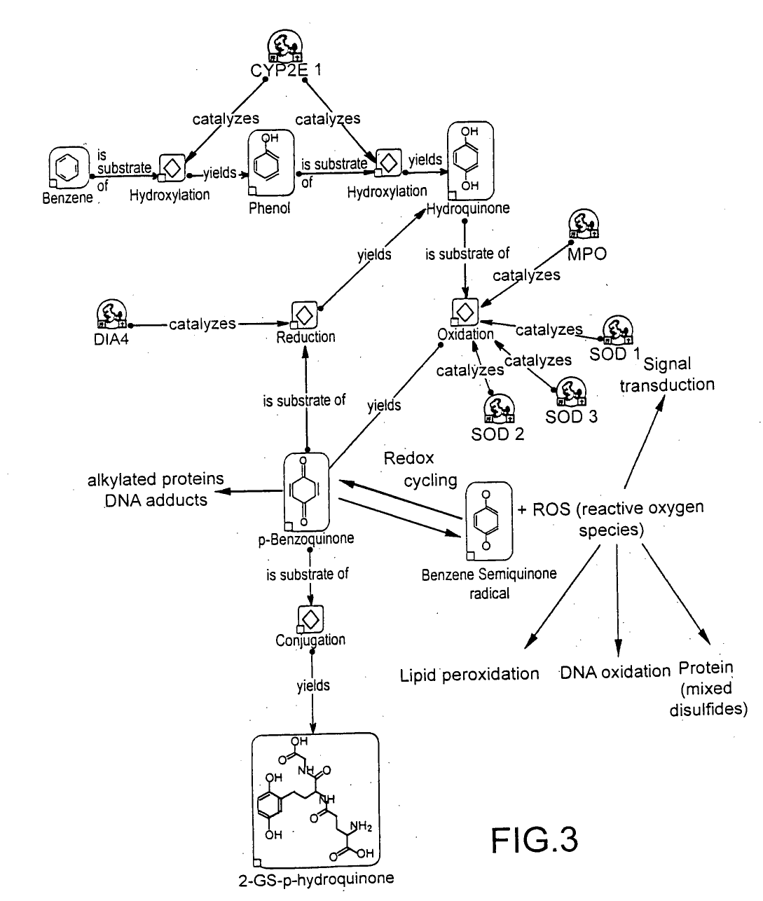

FIG. 3 shows a potential cytotoxic mechanisms of quinone type compounds with a selected pathway from benzene to para-benzoquinone being shown as example. Reactive quinones are known to alkylate proteins and from DNA adducts. Via redox cycling to semiquinone radicals reactive oxygen species (ROS) can be released with lead to lipid peroxidation etc. Enzymes involved in such pathways are selected for the assays described in FIG. 1.

The tables show:

Table 1: ET receptor antagonists, their indications and status. The receptor type with which the drug is interacting is given in brackets.

Table 2: Drugs with primary pulmonary hypertension as ADR.

Table 3: Candidate genes and ADRs of ET receptor antagonists. Nucleotide counting of SNPs and variants start from the ATG start codon. The altered nucleotide is given after the position.

Table 4: Oligonucleotide sequences used for personalized drug safety of patients with pulmonary hypertension. The altered position in comparison to the wildtype sequence is underlined. Nucleotide counting starts from the ATG start codon if not stated otherwise.

Table 5: Enzymes and detection methods used for drug profiling.

Table 6: Compounds and drugs, which are known substrates or inhibitors of liver enzyme variants and ADRs.

Table 7: Oligonucleotide sequences used for genotyping of patients. The altered position in comparison to the wildtype sequence is underlined. Nucleotide counting starts from the ATG start codon if not stated otherwise.

### EXAMPLES:

### A. Individual Drug Safety of Patients with Pulmonaryhypertension and Other Diseases Based on Allelic Variation

Biology of endothelins and their receptors:

Endothelins (ETs) consist of a family of multifunctional peptides, which have been implicated in numerous physiological and pathological conditions, like hypertension (Krum, H., Viskoper, R. J., Lacourciere, Y., Budde, M. & Charlon, V. The effect of an endothelin-receptor antagonist, bosentan, on blood pressure in patients with essential hypertension. Bosentan Hypertension Investigators. N Engl J Med 338, 784-790, 1998), pulmonary hypertension (Kohno, M. et al. Plasma immunoreactive endothelin-1 in experimental malignant hypertension. Hypertension 18, 93-100, 1991), acute renal failure (Shibouta, Y. et al. Pathophysiological role of endothelin in acute renal failure. Life Sci 46, 1611-1618, 1990), angina pectoris (Toyo-oka, T. et al. Increased plasma level of endothelin-1 and coronary spasm induction in patients with vasospastic angina pectoris. Circulation 83, 476-483, 1991), cardiac failure (Sakai, S. et al. Inhibition of myocardial endothelin pathway improves long-term survival in heart failure. Nature 384, 353-355, 1996); Schiffrin, E. L., Intengan, H. D., Thibault, G. & Touyz, R. M. Clinical significance of endothelin in cardiovascular disease. Curr Opin Cardiol 12, 354-367, 1997); Givertz, M. M. & Colucci, W. S. New targets for heart-failure therapy: endothelin, inflammatory cytokines, and oxidative stress. Lancet 352 Suppl 1, SI34-8, 1998; Krum, H. New and emerging pharmacological strategies in the management of chronic heart failure. Curr Opin Pharmacol 1, 126-133, 2001; Mulder, P., Richard, V. & Thuillez, C. Endothelin antagonism in experimental ischemic heart failure: hemodynamic, structural and neurohumoral effects. Heart Fail Rev 6, 295-300, 2001), disseminated intravascular coagulation, cancer (Norman, P. Atrasentan Abbott. Curr Opin Investig Drugs 3, 1240-1248, 2002) and others. Family members are ET-1, ET-2 and ET-3. They elicit biological responses by various signal transduction mechanisms, such as the G protein-coupled activation of phospholipase C and the activation of voltage-dependent Ca ²⁺ channels. Among the ET members, ET-1 has been studied most extensively since its discovery in 1988. ET-1 is synthesized via proteolytic cleavage of a large precursor molecule, pre-pro ET-1, which is catalyzed by the metalloproteinase, endothelin converting enzyme (ECE).

ETs perform their physiological effects via two receptors, ET receptor A (ETA) and ET receptor B (ETB). Both are G-protein coupled transmembrane receptors found in both vascular and nonvascular tissues. The affinity of ET towards ET_{A} is ET-1>ET-2>ET-3, whereas ET_{B} shows no selective affinity for any of the ET subtypes (Sakamoto, A. et al. Cloning and functional expression of human cDNA for the ETB endothelia receptor. Biochem Biophys Res Commun 178, 656-663, 1991).

ET receptors are G-protein-coupled and therefore, contain seven transmembrane regions. The three-dimensional structure of ET_{A} has been constructed by homology modeling. The principal interaction sites with ET lie on one side of a helix (Orry, A. J. & Wallace, B. A. Modeling and docking the endothelia G-protein-coupled receptor. Biophys J 79, 3083-3094, 2000). The binding site for BQ123, an ET_{A} antagonist, has been modeled with the help of ET_{A} mutants (Bhatnagar, S. & Rao, G. S. Molecular modeling of the complex of endothelin-1 (ET-1) with the endothelia type A (ET(A)) receptor and the rational design of a peptide antagonist. J Biomol Struct Dyn 17, 957-964, 2000). Based on these models, rational designs of peptide antagonists can be proposed.

Pharmacology:

ET_{A} is the mediator of the diseases treated with ET receptor antagonists. Among other roles the function of ETB is to clear ET-1. Therefore, the main interest is to develop ETA antagonists (Wu, C. Recent discovery and development of endothelia receptor antagonists. Exp. Opin. Ther. Patents 10, 1653-1668, 2000 and Table 1). ET receptor antagonists such as bosentan and tezosentan are used in the treatment of pulmonary hypertension and congestive heart failure (Donckier, J. E. Therapeutic role of bosentan in hypertension: lessons from the model of perinephritic hypertension, Heart Fail Rev 6, 253-264, 2001; Weber, C., Gasser, R. & Hopfgartner, G. Absorption, excretion, and metabolism of the endothelia receptor antagonist bosentan in healthy male subjects, Drug Metab Dispos 27, 810-815, 1999). Each new compound displays so far unpredictable ADRs(Galie, N., Manes, A. & Branzi, A. The new clinical trials on pharmacological treatment in pulmonary arterial hypertension, Eur Respir J 20, 1037-1049, 2002). Primary pulmonary hypertension is characterized by persistent elevation of pulmonary artery pressure without any known cause. Without treatment the mean age of survival is 2.8 years, but with treatment patients can survive for more than 10 years (Berkowitz, D. S. & Coyne, N. G. Understanding primary pulmonary hypertension, Crit Care Nurs Q 26, 28-34, 2003).

Diseases caused by obesity are treated with ET receptor antagonists. These diseases include those frequently associated with obesity such as hypertension, type 2 diabetes, hyperlipidemia, chronic kidney failure, arteriosclerosis and gout (hunter, K. & Kirchengast, M. Method for combating obesity. U.S. Pat. No. 6,197,780, 2001). Patients with hypertension present increased vascular levels of pre-pro ET-1 mRNA(Iglarz, M. & Schiffrin, E. L. Role of endothelin-1 in hypertension. Curr Hypertens Rep 5, 144-148, 2003).

ET_{A} receptor and mixed ETA/ATB receptor antagonists are used for treatment of patients with heart failure(Spieker, L. E. & Luscher, T. F. Will endothelin receptor antagonists have a role in heart failure? Med Clin North Am 87, 459-474, 2003).

Elevated plasma levels of ET-1 have been detected in patients with various solid tumors and ET-1 seems to act as a survival factor (Grant, K., Loizidou, M. & Taylor, I. Endothelin-1: a multifunctional molecule in cancer. Br J Cancer 88, 163-166, 2003). Atrasentan (Table 1) is under development for the treatment of prostate cancer and potential treatment of other cancer types.

For some of the ET receptor antagonists (Table 1) associated ADRs have already been identified. Bosentan can lead to liver injury (Fattinger, K. et al. The endothelin antagonist bosentan inhibits the canalicular bile salt export pump: a potential mechanism for hepatic adverse reactions. Clin Pharmacol Ther 69, 223-231, 2001). The ET_{B} antagonist BQ-788 showed a decrease of cerebral blood flow in rats (Chuquet, J. et al. Selective blockade of endothelin-B receptors exacerbates ischemic brain damage in the rat. Stroke 33, 3019-3025, 2002). Atrasentan led to headache, peripheral edema and rhinitis in a phase II study on prostate tumor progression in men (Carducci, M. A. et al. Effect of endothelin-A receptor blockade with atrasentan on tumor progression in men with hormone-refractory prostate cancer: a randomized, phase II, placebo-controlled trial. J Clin Oncol 21, 679-689, 2003).

Molecular biology:

Three genes coding for different ETs (ET-1, ET-2 and ET-3) have been identified (moue, A. et al. The human endothelin family: three structurally and pharmacologically distinct isopeptides predicted by three separate genes. Proc Natl Acad Sci U S A 86, 2863-2867, 1989). The functions of ETs are mediated by two receptors. The human ET_{A} cDNA codes for a 427 amino acid protein and the human ET_{B} for a 442 amino acid protein, respectively. ET_{A} and ET_{B} are known targets for bosentan and other drugs used in patients showing pulmonary hypertension. Alternative splice variants have been verified for both, ET_{A} and ET_{B}. ET_{A} MRNA is mainly expressed in the central nervous system, heart and lung-, ET_{B} MRNA mainly in brain, kidney and lung but not in vascular smooth muscle cells. Human ETA has been localized to chromosome 4. Several promoter elements of the ETA gene have been identified. Mutations in the ET_{B} gene are associated with Hirschsprung disease. Allelic and protein variants of ET_{B} are summarized in FIG. 1. For ET_{A} only one variant has been described so far (see Table 4).

Pharmacogenomics:

To assess the origins of individual variations in disease susceptibility or drug response, pharmacogenomics uses the genomic technologies to identify polymorphisms within genes that are part of biological pathways involved in disease susceptibility, etiology, and development, or more specifically in drug response pathways responsible for a drug's efficacy, tolerance, or toxicity, including but not limited to drug metabolism cascades.

Some alleles, SNPs or mutations can be linked to the occurrence of certain diseases or ADRs. But it is the combination of many SNPs and alleles that determines a person's susceptibility. DNA hybridization techniques are used to distinguish alleles involved in pulmonary hypertension and related diseases. The result is a genetic profile displaying the patient's susceptibility to ADRs. Genes that have allelic variants, which play a role in pulmonary hypertension, are summarized below.

Candidate genes; pulmonary hypertension:

Described SNPs of the human ET_{A} and ET_{B} genes: All variants and SLAPS of ET receptor genes are promising candidates, which predict the outcome of the treatment with ET receptor antagonists (Table 4). In addition, SNPs in genes leading to ADRs by applying drugs against pulmonary hypertension should be available soon (Table 2). ABCC2 (MRP2, cMOAT): Bosentan alters canalicular bile formation via ABCC2-mediated mechanisms in rats (Fouassier, L. et al. Contribution of mrp2 in alterations of canalicular bile formation by the endothelin antagonist bosentan. J Hepatol 37, 184-191, 2002).

Bsep (ABCB 11): ATP-binding cassette transporter of the multidrug resistance protein family. Bosentan can induce cholestatic liver injury through inhibition of Bsep-mediated canalicular bile salt transport.

AGTRl (Angiotensin II receptor type 1): Endogeneous angiotensin II induced cardiac fibrosis involves both, ET receptors and AGTR1. Dual antagonists of ET receptors and AGTR1 have been synthesized and specific antagonists of these receptors show combined effects. Several other interactions of ET and angiotensin II have been described.

The candidate genes are summarized in Table 3. Further candidate genes, which fit into the profile and will be identified in the future, will then be added.

### B. High throughput Analysis of Compounds and Drugs for Guinone and Radical Formation and Correlation to Genotypes

Drug metabolism:

Although many mechanisms of liver toxicity are known, there are still no methods available to design non-hepatotoxic drugs rationally, nor to screen compounds for reaction products with liver enzymes in a parallelized high throughput assay. The application is based on immobilized enzymes (e.g. CYPs, DIAs) so screen compounds and drugs for quinones, related substances and radical formation. The invention uses enzyme assays adapted for this purpose and combines it with genotyping.

Since one enzyme chip has to be designed for each assay type, in some cases it is of an advantage to put the drug candidates onto the chip surface. They can be either attached or in soluble form (microfluidics, lab-on-a-chip technology).

The reactions leading to quinone and radical formation are shown in FIG. 3. Detoxification begins with oxidation reactions catalyzed by CYPs. We analyse all drug-responsive molecules and their polymorphisms and mutations. This includes phase I enzymes, phase II enzymes, transporters, receptors, ion channels and transcrition factors involved in drug responses. For each enzyme a specific assay will be adapted to a miroarray format.

Pharmacogenomics:

For almost all enzymes polymorphisms are described and a big effort is currently undertaken to identify all polymorphisms in the human genome and correlate them to drug metabolism and disease. One of the many examples is the polymorphism at amino acid position 187 of NQ 01 which may correlate with susceptibility to cancer. This variant shows a diminished NQ 01 activity and thereby increases the risk of leukemia as a result of chemotherapy.

Now, the invention will be described in general terms.

Basic technique:

A defined clinical endpoint or disease is selected. All known genes allelic variants and SNPs, which are involved in producing this endpoint, are listed and analyzed. Then all drugs used in the treatment of this clinical endpoint are listed and analyzed for ADRs. The proteins interacting with the drugs are identified if known. The genes (called “candidate genes”) and allelic variants thereof causing these ADRs are collected. All this information and related information (e.g. pharmacokinetic data) is stored in a system that uses one format (SafeBase™, TheraSTrat). When 15 new candidate genes or SNPs are described they will be added.

A general strategic overview is presented in FIG. 1. We use a combination of different pattern analysis tools (pats) down to the single molecule level. All kinds of nucleic acid changes such as SNPs, deletions, insertions, amplifications, rearrangements, etc. are analyzed in patient DNA or RNA. Antibody pats are either used for the detection of changes in protein expression or protein binding or for the detection of disease-related non-protein antigens. In a similar way protein pats are used to measure changes in protein-protein interaction. Drug candidates or proteins are either used as pat probe or pat target, respectively. Chemical and biological similarity searches with many parameters will supplement ADR profiling and drug development.

Sequence regions which are responsible for ADRs are identified in the candidate genes. Oligonucleotide sequences containing the SNP or altered position are selected in the genomic DNA or cDNA sequences for diagnostic DNA hybridizations. Such sequences can be derived from the coding region, the 5′ or 3′ non-translated region, the intron or the promoter region. Even far upstream-located enhancer or silencer sequences are included. Wildtype sequences of the corresponding region are always included as controls. Oligonucleotides are usually 15 by long with the mutated or polymorphic nucleotide in the middle. Small deletions or insertions can also be analyzed. The oligonucleotide sequences are analyzed for hair-pin formation, melting temperature, inter- and intra hybridization. If a sequence is sub-optimal regarding theses parameters it will be adjusted by moving one to three nucleotides along the sequence in either direction or by prolongation of one end by one to three nucleotides.

SNP detection methods can either be oligonucleotide macro- or micro-arrays (GeneChips, Affymetrix, USA; MIP™, ParAllele BioScience, USA; Nanogene, USA), or oligonucleotides coupled to beads (e.g. Teflon beads; BeadArray™, Illumina, USA; SmartBead Technology, USA) or mass spectrometry (e.g. “matrix-assisted laser-desorption ionization” with “time of flight” analysis (MALDI-TOF (MassARRAY™, Sequenom, USA)).

The oligonucleotide sequences will be connected to specific ADRs with genomic DNA samples of patients under defined treatments in clinical studies. Below we describe an example for pulmonary hypertension patients. The protocol is easily adapted for other diseases. Our approach can be extended to personalized heart failure treatment.

Liver enzymes and their variants are immobilized onto solid surfaces. Compounds and drugs are used as targets for these immobilized enzymes to detect formation of quinones, quinoneimins and radicals with a large number of different enzymes in parallel. The detection is performed with assays adapted to the surfaces. This yields a profile of a compound or drug describing which enzymes and their allelic variants are leading to potentially harmful intermediates in the liver. All these data are stored in a database (SafeBase™), which will help in identifying new drug targets. In addition, profiles of the allelic variants of liver enzymes are collected by SNP genotyping and stored in the same database. The final outcome is a personal drug profile. Some drugs can only be prescribed to patients with a certain allelic profile.

### A. Individual Drug Safety of Patients with Pulmonary Hypertension and other Diseases Based, on Allelic Variation

ADRs of ET receptor antagonists:

In addition to the already described ADRs of the ET receptor antagonists listed in Table 1 a general study of ADRs mediated by ET receptor antagonists has to be performed.

ET receptor allelic variants that are known to be involved in inducing defined ADRs are shown in Table 3. Moreover, the ADRs have to be correlated with specific patterns of SNPs, variations and mutations in the candidate genes shown in Tables 3 and 4. This profile list has to be continuously updated and adjusted with the advancement of new technologies.

Candidate gene selection:

In order to identify new candidate genes and their SNPs and mutations in addition to the ET receptor genes we analyze drug-induced pathways that lead to pulmonary hypertension as ADR (Table 2). As can be seen, diet pills induce pulmonary hypertension. The responsible genes and alleles will be identified.

The genes, alleles and SNPs, which code for proteins that interact with ET receptors antagonists are analyzed for ADRs (Table 3). A major part of the invention is to detect specific profiles of allele and SNP combinations that lead to defined ADRs.

Sequence selection:

The selected oligonucleotide sequences, which contain the SNPs and positions of small deletions or insertions, are shown in Table 4. These sequences and other sequences of SNPs coming in the future are used for the hybridization experiments to determine the ADR profile of pulmonary hypertension patients. The invention is based on the combined analysis of these SNPs and variants in patient DNA samples.

Result:

ADRs that can occur in patients, which have to undergo treatment of pulmonary hypertension, are predictable. The allele profile will tell the patient which types of ADRs he or she has to expect under certain treatments. According to this knowledge an optimal personal therapy protocol can be designed.

### C. High throughput Analysis of Compounds and Drugs for Quinone and Radical Formation and Correlation to Genotypes

Detection of enzymatic activities:

We examine the products and kinetics of enzymes and their variants with catalytic mechanisms to attack a variety of moieties and which therefore produce different types of metabolites. Among enzymes with a single type of reaction like N-actetyltransferases we compare the activities of different enzyme variants. The drug libraries are from pharmaceutical companies (e.g. Roche, Switzerland).

Enzymes, isozymes and variants are attached to solid surfaces (e.g. XNA on gold (ThermoHybaid, USA, Pavlickova, P., Knappik, A., Kambhampati, D., Ortigao, F. & Hug, H. Microarray of recombinant antibodies using a streptavidin sensor surface self-assembled onto a gold layer, Biotechniques 34, 124-130, 2003), PWG-chips (Zeptosens, Switzerland, Pawlak, M. et al. Zeptosens' protein microarrays: a novel high performance microarray platform for low abundance protein analysis. Proteomics 2,-3.83-393, 2002), Ciphergen biosystems, USA; Zyomics, USA; Clondiag, Germany). The enzyme classes from which members are used for immobilization are CYPs, dehydrogenases and reductases, flavin-containing monooxygenases, hydrolases, methyltransferases, sulfotransferases, glucuronyltransferases, N-acetyltransferases, acyl-coenzyme A synthetases, glutathione S-transferases and phosphotransferases. Human purified native and recombinant enzymes are commercially available (e.g. Research Diagnostics Inc, USA; PanVera-InvitrogenTM, USA; BD GentestTM, USA).

Alternatively the candidate drugs are put onto the solid surface. There are several recent developments that use micro-chambers to hold the substance in solution for the reactions (e.g. Advalytix, Germany; Advion BioSciences, USA; BioForce Nanosciences Inc, USA; BioTrace, USA; Caliper Technologies, USA). Enzymes are added either single or in combinations into the chambers and the reaction products are detected.

Enzyme activity is measured with fluorescently labeled substrates directly on the surface in aqueous solution (e.g. Vivid®CYP450, PanVera, USA). Examples with described detection methods are listed in Table 5.

The enzyme activity profile is useful for preclinical drug screening and diagnosis when lysates from liver biopsies are tested. In this way a huge amount of pharmacokinetic data are obtained due to parallelization. This leads to a more rapid prodrug and soft drug design. In addition many combinations for potential drug-drug interactions can be studied.

Drugs interacting with liver enzymes:

Drugs, which are substrates or inhibitors of liver enzymes, are examined whether defined enzyme variants are responsible for the effects. If possible, ADRs are also connected to the enzyme variants (Table 6).

Genotyping of liver enzymes:

The enzyme activity profiles have to be correlated with genotypes. If the genotype of the liver enzyme variants is known it can be predicted which drugs and how the person metabolizes drugs. The effect of combinations of drugs will also be predictable. The genotypes are related to ADRs with SafeBase™ (TheraSTrat).

The DNA sequences coding for enzyme variants are available from public databases (e.g. EMBL). Oligonucleotides containing the variable position in the middle are usually 15 by long (Table 7). Again, sequences are shifted if hair pin or dimer formation have to be inhibited or if the melting temperature has to be adapted. SNP detection methods are as described above and the obtained profiles are evaluated with SafeBase™ (TheraSTrat). Further candidate genes and their alleles will be added as soon as they are identified.

Personalized drug safety:

The result of our invention is the prediction of ADRs that can occur in patients, which have to undergo treatment of disease. The allele profile will tell the patient which types of ADRs she or he has to expect under certain treatments. According to this knowledge an optimal personal therapy protocol can be designed.

TABLE 1
ET receptor antagonists, their indications and status. The receptor
type with which the drug is interacting is given in brackets. m, on
the market, exp, in the experimental phase. PPH, primary
pulmonary hypertension; AHF, acute heart failure; CHF,
congestive heart failure; CAD, coronary artery disease;
H, hypertension; IIND, ischemia-induced neuronal
degradation; ?, not known at present.

| ET receptor antagonist | Indication | Status |
|---|---|---|
| Bosentan (ET_{A}/ET_{B}) | PPH, CHF | m |
| Tezosentan (ET_{A}/ET_{B}) | PPH, AHF | III |
| BQ-123 (ET_{A}) | CHF, AHF | exp |
| BQ-788 (ET_{B}) | Stroke | ? |
| Sitaxsentan (ET_{A}) | PPH, CHF | II/III |
| BMS-193884 (ET_{A}) | CHF | II |
| BMS-207940 (ET_{A}) | Diabetes | ? |
| SB-209670 (ET_{A}/ET_{B}) | Renal failure, IIND, H | II |
| Enrasentan (ET_{A}/ET_{B}) | CHF, H | II |
| SB-209598 | ? | ? |
| TAK-044 (ET_{A}/ET_{B}) | CAD, H | II |
| PD-156707 | ? | ? |
| L-749329 (ET_{A}) | ? | ? |
| L-754142 (ET_{A}) | ? | ? |
| Atrasentan (ET_{A}) | CHF, prostate cancer | III |
| A-127772 (ET_{A}) | ? | ? |
| A-206377 | ? | ? |
| A-182086 (ET_{A}/ET_{B}) | ? | ? |
| EMD-94246 (ET_{A}) | CHF, H | ? |
| EMD-122801 | ? | ? |
| ZD-1611 (ET_{A}) | PPH, obstructive lung disease | ? |
| AC610612 | ? | ? |
| Darusentan (ET_{A}/ET_{B}) | CHF, H | II |
| T-0201 | ? | ? |
| J-104132 (ET_{A}/ET_{B}) | CHF, H | II |
| STR2 | ? | ? |
| STR3 | ? | ? |
| RO46-2005 (ET_{A}/ET_{B}) | ? | ? |
| RO61-1790 (ET_{A}) | Subarachnoidal hemorrhage | ? |

TABLE 2
Drugs with primary pulmonary hypertension as ADR. Other
cases are expected to occur in the future. These will be
then integrated in ADR profiling.

| Drug | Synonym | Usage |
|---|---|---|
| Dexfenfluramine | Redux ® | Anorexigen |
| Fenfluramine Hydrochloride | Ponderal ®, Pondimin ® | Anorexigen |
| Phendimetrazine | Plegine ® | Anorexigen |
| Phentermine | | Anorexigen |

TABLE 3
Candidate genes and ADRs of ET receptor antagonists. Nucleotide
counting of SNPs and variants start from the ATG start codon. The
altered nucleotide is given after the position. Asterisks indicate
allelic variants. More correlations are expected to occur in the
future. The list is continuously updated. Wt, wildtype

| Gene | SNP or variant | ADR |
|---|---|---|
| ET_{A} | EDNRA*-2316 > A | Migraine |
| | H232H(C/T) | Shorter survival of dilated |
| | | cardiomyopathy |
| | Wt? | Headache |
| | Wt? | Peripheral edema |
| | Wt? | Rhinitis |
| ET_{B} | | |
| ABCC2 (MRP2) | | Alteration of canalicular bile |
| | | formation |
| Bsep | | Cholestatic liver injury |
| AGTR 1 | | |

TABLE 4

Oligonucleotide sequences used for personalized
drug safety of patients with pulmonary hyper-
tension. Nucleotide counting starts from the
ATG start codon if not stated otherwise.
Asterisks indicate allelic variants. X > Y
means that nucleotide X is exchanged by nucle-
otide Y. The altered position in comparison to
the wildtype sequence is underlined. The first
number after an intron (IVS) gives the number
of the intron, the second number gives the po-
sition of the mutation starting to count from
the 5′ splice site in that intron. Genes and
variants represent the present state. The list
is continuously updated with new variants and
candidate genes. UTR, non-translated region;
del, deletion; ins, insertion; ?, sequence not
available at present.

| | | Oligonucleotide |
|---|---|---|
| Gene | SNP or variant | sequence |
| ET_{A} | EDNRA*−231G > A | CCCAGGAAGTTTTCT |
| Gene | SNP or variant | Oligonucleotide |
| | | sequence |
| ET_{B} | EDNRB*−26G > A | GCCACCAGACGGCCT |
| | EDNRB*169G > A | GCCCAAGAGTTCCAA |
| | EDNRB*325T > C | TGTGTCCTGCCTTGT |
| | EDNRB*548C > G | ACAGAAAGGCTCCGT |
| | EDNRB*556G > A | CTCCGTGAGAATCAC |
| | EDNRB*678G > T | TGATTTGTGTGGTCT |
| | EDNRB*757C > T | TTATCTGTGAATCTG |
| | EDNRB*824G > A | AAAAGATTAGTGGCT |
| | EDNRB*828G > T | TTGGTGTCTGTTCAG |
| | EDNRB*878ins | TTTTTTATTACACTA |
| | EDNRB*914G > A | AGAAAAATGGCATGC |
| | EDNRB*928G > A | GCAGATTACTTTAAA |
| | EDNRB*955C > T | GCAGAGATGGGAAGT |
| | EDNRB*1132delA | CTTCACTG_ATTCCTG |
| | EDNRB*1148C > T | CATTAACCTAATTGC |
| | EDNRB*1170C > A | TGGTGAGAAAAAGAT |
| ABCC2 | ABCC2*−24C > T | GAGTCTTTGTTCCAG |
| | ABCC2*12496 > A | GTACACCATTGGAGA |
| | ABCC2*2302C > T | GAAGCAGTGGATCAG |
| | ABCC2*2366C > T | CCCCTGTTTGCAGTG |
| | ABCC2*3196C > T | ? |
| | ABCC2*3972C > T | GTGACATTGGTAGCA |
| | ABCC2*4145A > G | ATCCCCCGGGACCCC |
| | ABCC2*4348G > A | GGGCAGGACTCTGCT |
| | ABCC2*IVS13 + 2T > A | TCCAGGAAGGTCGGC |
| | ABCC2*IVS15 + 2T > C | |
| | ABCC2*IVS18 + 2T > C | GGCAAGGCGAGAATC |
| Bsep | ABCB11*890A > G | GGTGGTGGGAAAAGA |
| | ABCB11*908delG | GTTGAAA_GTATGAGA |
| | ABCB11*1381A > G | AGCTGGAGAAAGTAC |
| | ABCB11*1445A > G | ACCGTGGGTGGCCAT |
| | ABCB11*1723C > T | CCTCATCTGAAATCC |
| | ABCB11*2944G > A | TATTTACAGATTCTG |
| | ABCB11*3169C > T | GCTGGACTGACAACC |
| | ABCB11*3457C > T | GTTCCTCTGCTCAAA |
| | ABCB11*3767ins | AAAAGACCGGTGCAG |
| | ABCB11*3803G > A | GAGGGTCAGACCTGC |
| AGTR1 | AGTR1*142T > G | AAACAGCGTGGTGGT |
| | AGTR1*867T > G | CCATTTGGATAGCTT |
| | AGTR1*1006A > C | AATGAGCCCGCTTTC |
| | AGTR1*3′UTRA < C | AATGAGCCTTAGCTA |

TABLE 5
Enzymes and detection methods used for drug profiling. The list
is not complete and is continuously updated.

| Enzyme or variant | In vivo reaction | In vitro detection |
|---|---|---|
| NQOI (DIA4) | Reduction of quinone | MTT assay |
| NQOI*2 | No activity | MTT assay |
| (DIA4*609C>T) | | |
| NQ02 | | |
| CYP 1A1 | 7-deethylation of | 7-deethylation of |
| | ethoxyresorufin | ethoxyresorufin |
| CYP 1A2 | Substrates are basic | Release of resorufin from |
| | planar molecules | methoxyresorufin |
| CYP 1B1 | | 7-deethylation of |
| | | ethoxyresorufin |
| CYP 2A6 | 7-hydroxylation of | 7-hydroxylation of |
| | coumarin | coumarin |
| CYP 2B4 | | Release of re sorufin |
| | | from pentoxyresorufin |
| CYP 2B6 | 7-deethylation of | 7-deethylation of |
| | ethoxycoumarin | ethoxycoumarin |
| CYP 2C8 | 6α-hydroxylation of | 6α-hydroxylation |
| | paclitaxel | of paclitaxel |
| CYP 2C9 | Generation of ROS | Hydroxylation of |
| | | diclofenac |
| CYP 2C9*2 | | 4-hydroxylation of |
| | | diclofenac |
| CYP 2C18 | | 4-hydroxylation of |
| | | diclofenac |
| CYP 2C 19 | | Hydroxylation of |
| | | Mephenytoin |
| CYP 2D6 | Formation of R- and S- | Hydroxylation of |
| | norfluoxetine, 4- | bufuralol |
| | hydrolation | |
| | of debrisoquine | |
| CYP 3A4 | 6β-hydroxylation of | 6β-hydroxylation of |
| | testosterone, oxidation of | testosterone |
| | nifedipine, N- | |
| | demethylation | |
| | of dextrometorphan and | |
| | erythromycin | |
| MPO | | 4-mrophenyl -phosphate |
| SOD | | WST-1 + H₂0₂ |
| EPHX1 | | |
| DHDD | | |
| AKR i | | |
| GST | Conjugation of toxicants | CDNB + GSSG |
| | to GSH | |
| BVRA | Reduction of biliverdin | Reduction of biliverdin |

TABLE 6
Compounds and drugs, which are known substrates or inhibitors
of enzyme variants and ADRs. The list is not complete is
continuously updated.

| Compound | Enzyme | |
|---|---|---|
| or drug | or variant | ADR |
| Cytostatics | NQOI *2 | Leukemia |
| | (DIA4*609C>T) | |
| Halothane | | Hepatitis |
| Benzene | see FIG. 3 | |
| Menadione | NQO1 | hemolytic anemia |
| Ubiquinone/Vitamine E | | Regeneration of |
| | | antioxidants is missing |
| Troglitazone | PST1A3 | Hepatotoxicity |
| Troglitazone I | Bax, JNK | Apoptosis |

TABLE 7

Oligonucleotide sequences used for genotyping
of patients. The altered position in comparison
to the wildtype sequence is underlined. Nucle-
otide counting starts from the ATG start codon
if not stated otherwise. Asterisks indicate
allelic variants. X > Y means that nucleotide X
is exchanged by nucleotide Y. The altered po-
sition in comparison to the wildtype sequence
is underlined. The first number after an intron
(IVS) gives the number of the intron, the
second number gives the position of the muta-
tion starting to count from the 5′ splice site
in that intron. The list contains representa-
tive examples and is not complete. SNPs occur-
ring in different variants are only put once
onto the surface. Genes and variants represent
part of the present state. The list is contin-
uously updated with new variants and candidate
genes. UTR, non-translated region; del, dele-
tion; ins, insertion.

| | | Oligonucleotide |
|---|---|---|
| Gene | Variant | sequence |
| CYP2C9 | CYP2C9*2/430C > T | TGAGGACTGTGTTCA |
| | CYP2C9*3/1075A> C | GAGATACCTTGACCT |
| CYP2C19 | CYP2C19*2A/99C > T | CTGGCCCTACTCCTC |
| | CYP2C19*2A/681G > A | TTTCCCCAGGAACCC |
| | CYP2C19*2A/991A > G | CGTGTCGTTGGCAGA |
| | CYP2C19*2A/990C > T | GAACGTGTTATTGGC |
| | CYP2C19*3/636G > A | CCCCCTGAATCCAGA |
| | CYP2C19*3/991A > G | CGTGTCGTTGGCAGA |
| | CYP2C19*3/1251A > C | AAGGTGGCAATTTTA |
| | CYP2C19*4/1A > G | AACTTCAGTGGATCC |
| | CYP2C19*4/99C > T | CTGGCCCTACTCCTC |
| | CYP2C19*4/991A > G | CGTGTCGTTGGCAGA |
| | CYP2C19*SA/1297C > T | AGGAAAATGGATTTG |
| CYP2D6 | CYP2D6*2A/−1584C > G+ | AAGAACCGGGTCTCT |
| | CYP2D6*2A/−1235A > G+ | AAAAAGGATTAGGCT |
| | CYP2D6*2A/−740C > T+ | TGTGTGCTCTAAGTG |
| | CYP2D6*2A/−678G > A+ | TTCTGCATGTGTAAT |
| | CYP2D6*2A/1661G > C+ | TCTCCGTCTCCACCT |
| | CYP2D6*2A/2850C > T+ | GAACCTGTGCATAGT |
| | CYP2D6*2A/4180G > C | CTGGTGACCCCATCC |
| | CYP2D6*3A/2549A > del | TGAGCAC_GGATGACC |
| | CYP2D6*4A/4180G > C+ | CTGGTGACCCCATCC |
| | CYP2D6*4A/1846G > A+ | ACCCCCAAGACGCCC |
| | CYP2D6*4A/1661G > C+ | TCTCCGTCTCCACCT |
| | CYP2D6*4A/974C > A+ | GCGAGGCGATGGTGA |
| | CYP2D6*4A/997C > G+ | GGACACGGCCGACCG |
| | CYP2D6*4A/984A > G+ | GTGACCCGCGGCGAG |
| | CYP2D6*4A/100C > T | ACGCTACTCACCAGG |
| | CYP2D6*6A/1707T > del | TGGAGCAG_GGGTGAC |
| | CYP2D6*7/2935A > C | GATCCTACCTCCGGA |
| | CYP2D6*8/1661G > C+ | TCTCCGTCTCCACCT |
| | CYP2D6*8/1758G > T+ | CCACTCCTGTGGGTG |
| | CYP2D6*8/2850C > T+ | GAACCTGTGCATAGT |
| | CYP2D6*8/4180G > C | CTGGTGACCCCATCC |
| | CYP2D6*9/2613-2615 | AGAGATGG_{——}AGGTGAGA |
| | delAG | |
| | CYP2D6*10A/100C > T | ACGCTACTCACCAGG |
| | CYP2D6*10A/1661G > C | TCTCCGTCTCCACCT |
| | CYP2D6*10A/41806 > C | CTGGTGACCCCATCC |
| | CYP2D6*11/883G > C+ | CTCTGCACTTGCGGC |
| | CYP2D6*11/16616 > C+ | TCTCCGTCTCCACCT |
| | CYP2D6*11/2850C > T+ | GAACCTGTGCATAGT |
| | CYP2D6*11/4180G > C | CTGGTGACCCCATCC |
| | CYP2D6*12/124G > A+ | ACTGCCCAGGCTGGG |
| | CYP2D6*12/1661G > C+ | TCTCCGTCTCCACCT |
| | CYP2D6*12/2850C > T+ | GAACCTGTGCATAGT |
| | CYP2D6*12/41806 > C | CTGGTGACCCCATCC |
| | CYP2D6*14A/100C > T+ | ACGCTACTCACCAGG |
| | CYP2D6*14A/1758G > A+ | CCACTCCTGTGGGTG |
| | CYP2D6*14A12850C > T+ | GAACCTGTGCATAGT |
| | CYP2D6*14A/4180G > C | CTGGTGACCCCATCC |
| | CYP2D6*15/138insT | CAACCTGTCTGCATG |
| | CYP2D6*17/1023C > T+ | CCCATCATCCAGATC |
| | CYP2D6*17/16386 > C+ | CGCGTGGCGCGAGCA |
| | CYP2D6*17/2850C > T+ | GAACCTGTGCATAGT |
| | CYP2D6*17/41806 > C | CTGGTGACCCCATCC |
| | CYP2D6*19/1661G > C+ | TCTCCGTCTCCACCT |
| | CYP2D6*19/2539- | AGCTGCT_{———}GAGCACA |
| | 2542delAACT+ | |
| | CYP2D6*19/2850C > T+ | GAACCTGTGCATAGT |
| | CYP2D6*19/41806 > C | CTGGTGACCCCATCC |
| | CYP2D6*20/1661G > C+ | TCTCCGTCTCCACCT |
| | CYP2D6*20/1973insG+ | CTCAGGAGGGGACTG |
| | CYP2D6*20/1978C > T+ | AGGAGGGATGAAGGA |
| | CYP2D6*20/1979T > C+ | AGGGACCGAAGGAGG |
| | CYP2D6*20/2850C > T+ | GAACCTGTGCATAGT |
| | CYP2D6*20/4180G > C | CTGGTGACCCCATCC |
| | CYP2D6*38/2587- | GAGACCT_{———}GAGGCCT |
| | 2590delGACT | |
| MDR1 | MDRI*3435C > T | AAGAGATTGTGAGGG |
| NAT2 | NAT2*5A1341T > C+ | GTGACCACTGACGGC |
| | NAT2*5A/481C > T | CTGGTACTTGGACCA |
| | NAT2*6A/282C > T+ | ATTTTTATATCCCTC |
| | NAT2*6A/5906 > A | GAACCTCAAACAATT |
| | NAT2*7A/857G > A | GGTGATGAATCCCTT |
| | NAT2*12A/803A > G | GTGCTGAGAAATATA |
| | NAT2*13/282C > T | ATTTTTATATCCCTC |
| | NAT2*14A/1916 > A | AGAAACCAGGGTGGG |
| | NAT2*17/434A > C | CAGCCTCCGGTGCCT |
| | NAT2*18/845A > C | GTGCCCACACCTGGT |
| NQO1 | DIA4*559C > T | CTTAGAATCTCAACT |
| DIAL | DIAl*129C > A | TCAAGTAACCGCTGC |
| | DIAl*1496 > A | ATCGACCAGGAGATC |
| | DIAl*173G > A | GACACCCAGCGCTTC |
| | DIAl*194C > T | GCCCTGCTGTCACCC |
| | DIAL*218T > C | CTGGGCCCCCCTGTC |
| | DIAl*229C > T | TGTCGGCTAGCACAT |
| | DIAl*250C > T | CTCGGCTTGAATTGA |
| | DIAl*287C > A | TATACACACATCTCC |
| | DIAl*316G > A | GGGCTTCATGGACCT |
| | DIAl*379A > G | AGGGAAGGTGTCTCA |
| | DIAL*382T > C | GAAGATGCCTCAGTA |
| | DIAl*434C > T | CGGGGCCTCAGTGGG |
| | DIAl*446T > C | GGGCTGCCGGTCTAC |
| | DIAl*478C > T | CGCCATCTGACCTGA |
| | DIAL*535G > A | GCATGATCACGGGAG |
| | DIAL*536C > T | TGATCGTGGGAGGGA |
| | DIAL*610T > C | ACACTGTGCGCCACC |
| | DIAl*611G > A | CTGTGTACCACCTGC |
| | DIAL*637G > A | CCAGACCAAGAAGGA |
| | DIAL*655C > T | CCTGCTGTGACCTGA |
| | DIAl*716T > G | TACACGCGGGACAGA |
| | DIAl*757G > A | GGGCTTCATGAATGA |
| | DIAl*815delTGA | GCTGGTGC_TGTGTGG |
| | DIAl*895delTTC | AGCGCTGC_GTCTTCT |
| SULTIAI | SULTIAI*2/638G > A | GTGGGGCACTCCCTG |
| (PST) | SULTIAI*3/667A > G | GGACTTCGTGGTTCA |
| | SULTIAI*4/11OG > A | CAGGCCCAGCCTGAT |
| | SULTIAI*5/436G > A+ | CCACATGACCAAGGT |
| | SULTIAI*5/542A > G+ | TGGTGGGGGCTGAGC |
| | SULTIAI*5/638G > A | GTGGGGCACTCCCTG |
| TPMT | TPMT*3A/460Cj > A + | TAGAGGAACATTAGT |
| | TPMT*3A/719A > G | AGTTATGTCTACTTA |
| | TPMT*3B/460G > A | TAGAGGAACATTAGT |
| | PMT*3C/719A > G | AGTTATGTCTACTTA |
| | TPMT*3D/292G > T+ | GATACAATAATTTTT |
| | TPMT*3D/719A > G+ | AGTTATGTCTACTTA |
| | TPMT*3D/460G > A | TAGAGGAACATTAGT |
| MPO | MPO*752T > C | TCACTCACGTTCATG |
| SOD | SOD1*26T > A | TGCGTGCAGAAGGGC |
| EPHXI | EPHXI*2/145C > T | CAGCATCTGCCCTTT |

1. Method for registration, identifying and processing of drug specific data, wherein genetic data and/or mRNA and/or protein expression data and/or protein activity data are correlated with each other, to drugs, chemicals beyond drugs, peptides, proteins, antibodies, nucleic acid based drugs, ADRs clinical endpoints and analyzed by chemical and biological similarity searches in order to yield a patient's individual ADR profile, wherein the said ADR profile is used for individual drug safety.

2. Method according to claim 1, wherein addressed drug candidates or immobilized liver enzymes are used for high throughput parallelized drug development and a special assay is adapted for each class of enzymes.

3. Method according to claim 2, wherein genetic data for correlation are only used.

4. Method according to claim 1, wherein the focus is on the protein activity side and wherein the activity of allelic variants of liver enzymes will be correlated to genotypes and ADRs according for example to Tables 5 and 7 and FIG. 3.

5. Method according to claim 1, wherein metabolite formation such as quinone and quinoneimine formation will be determined by screening a drug library according for example to FIG. 3.

6. A system for the identification of patients suited for defined therapies against pulmonary hypertension based on the method according to claim 1 and on prediction of ADRs arising during treatment of pulmonary hypertension, wherein oligonucleotide sequences derived from genes and alleles, whose products are targets for drugs used for the treatment of pulmonary hypertension or which by themselves play a role in the generation of pulmonary hypertension, are coupled to described surfaces (DNA-chips or -beads) wherein candidate genes and alleles are the endothelin receptors ETA and ETB or genes that are involved in ADRs caused by drugs used to treat pulmonary hypertension (ABCC2, Bsep, AGTR1, etc.) and wherein SNPs of the candidate genes are related to ADRs and clinical endpoints according for example to FIG. 2 and Table 4.10
